(19) United States
(12) Patent Application Publication
(10) Pub. No.: US 2026/0041933 A1
(43) Pub. Date: Feb. 12, 2026

(54) PROGRAMMABLE HEADSET FOR LOW LIGHT THERAPY AND VISUAL FUNCTION TESTING

(71) Applicant: Alcon Inc., Fribourg (CH)

(72) Inventor: Paul R. Hallen, Colleyville, TX (US)

(21) Appl. No.: 19/283,980

(22) Filed: Jul. 29, 2025

Related U.S. Application Data
(60) Provisional application No. 63/680,157, filed on Aug. 7, 2024.

(51) Int. Cl. A61N 5/067; A61N 5/06

(52) U.S. Cl. CPC A61N 5/067; A61N 5/0613; A61N 2005/0627; A61N 2005/0648; A61N 2005/0651; A61N 2005/0659; A61N 2005/0663

(57) ABSTRACT

A computer-implemented method of administering low light therapy. Embodiments include receiving data from one or more sensors. Embodiments include determining, based on the data, an identity of a user. Embodiments include administering, based on the identity of the user, a configured dose of low light therapy using one or more light sources.

### INTRODUCTION

Photo-bio-modulation (PBM) is a form of low light therapy that utilizes non-ionizing forms of light sources such as lasers, light-emitting diodes (LEDs), and/or broadband light, in the visible and near infrared spectrum. In some cases, PBM therapy may be performed on a patient's eye to treat certain ophthalmic diseases, such as retina disease. For example, a light source may be placed near or in contact with the eye, allowing the light energy (e.g., photons) to penetrate tissue and interact with chromophores in cells, resulting in photophysical and photochemical changes in diseased and damaged tissues. Such changes may accelerate wound healing and tissue regeneration, increase circulation, reduce acute inflammation, reduce pain, and/or help restore normal cellular function

PBM may be administered by a medical professional in an office. However, visiting a medical office for such treatments may be inconvenient for a patient, particularly if frequent doses are prescribed. Furthermore, existing techniques for administering PBM treatment may allow for misapplication of PBM treatment, such as unnecessarily large doses and/or doses that are not suited to a particular patient.

Accordingly, there is a need for improved techniques for optimizing administration of PBM dosing.

### SUMMARY

In certain embodiments, one general aspect includes a system for administering low light therapy. The system may include: one or more light sources configured to generate outgoing light across a range of wavelengths; one or more sensors configured to scan a biological attribute of a user; and one or more processors configured to execute instructions that cause the system to: determine, based on data from the one or more sensors, an identity of the user; and administer, based on the identity of the user, a configured dose of low light therapy using the one or more light sources.

In certain embodiments, another general aspect includes a computer-implemented method. The method may include: receiving data from one or more sensors; determining, based on the data, an identity of a user; and administering, based on the identity of the user, a configured dose of low light therapy using one or more light sources.

In certain embodiments, another general aspect includes a computer-program product including a non-transitory computer-usable medium having computer-readable program code embodied therein. The computer-readable program code is adapted to be executed to implement the computer-implemented method for administering low light therapy described above.

### BRIEF DESCRIPTION OF THE DRAWINGS

FIG. 1 illustrates an example of components related to administering low light therapy, in accordance with certain embodiments of the present disclosure.

FIG. 2 is a sequence diagram illustrating an example related to administering low light therapy, in accordance with certain embodiments of the present disclosure.

FIG. 3 illustrates an example of an electronic notification related to administering low light therapy, in accordance with certain embodiments of the present disclosure.

FIG. 4 illustrates an example of a process related to administering low light therapy, in accordance with certain embodiments of the present disclosure.

FIG. 5 illustrates an example of a computing device for administering low light therapy, in accordance with certain embodiments of the present disclosure.

### DETAILED DESCRIPTION

Recently, low light therapy such as photo-bio-modulation (PBM) for ophthalmic diseases has shown significant efficacy and safety. For example, low light therapy may be used to rejuvenate mitochondria in the retina by stimulation via specific wavelengths to treat diseases such as dry age-related macular disease (AMD), geographic atrophy, myopia, inherited retinal diseases, wet AMD, glaucoma, diabetic macular edema, diabetic retinopathy, and/or other degenerative eye diseases. Low light therapy may also be used for pre-surgical and/or post-surgical vitrectomy for a variety of causes (e.g., detachments, macular holes, and/or the like), such as to help prevent cell death or degradation of function. Currently, such low light therapy is administered via specialized equipment in a clinical setting, such as in a medical office. Existing devices for administering such treatment are overseen by medical professionals, as these devices may enable misapplication of low light therapy if used incorrectly. For example, existing devices, if not properly overseen, may allow unnecessarily large doses of low light therapy to be administered or may enable administration of a dose intended for one patient to another patient. However, it may be inconvenient for a patient to visit a medical office for every dose of low light therapy, particularly when frequent doses are prescribed. Thus, in some cases, patients may skip doses due to this inconvenience.

Aspects of the present disclosure overcome these challenges through a headset that is configured to automatically regulate and facilitate administration of low light therapy to particular patients in a programmable manner. In certain aspects, a headset may be configured to receive low light therapy dosing information for a particular patient, such as from a remote system associated with a medical professional. The headset may use one or more sensors to confirm an identity of a patient, such as via a retina scan, iris scan, fingerprint reading, and/or the like. The one or more sensors may be part of the headset and/or may be part of a separate device such as a mobile phone. Upon confirming the identity of a patient, the headset may administer a dose of low therapy to the patient according to the dosing information (e.g., received from a medical professional) for that patient.

The headset may prevent any administration of low light therapy beyond that prescribed in the dosing information for a particular patient, such as only administering low light therapy when a dose is due, only administering the amount prescribed for the dose, and only administering such therapy after confirming the identity of the particular patient. Furthermore, the headset or an associated component may generate notifications when doses are due, such as transmitting such notifications to a separate device (e.g., mobile phone) associated with a patient.

According to certain aspects, the headset may be configured to perform one or more ophthalmic tests on a patient, such as before and/or after administration of one or more doses of low light therapy. For example, the headset may include hardware (e.g., one or more light sources and/or sensors) that enables performing one or more ophthalmic tests such as visual acuity, contrast sensitivity, dark adaptation, optical coherence tomography (OCT), micro-perimetry, scanning laser ophthalmoscopy, multi-spectral imaging (MSI), and/or the like. The headset may be configured to transmit results of performing one or more such ophthalmic tests to a remote system (e.g., associated with a medical professional or another device associated with the user), such as for use in determining low light therapy dosing information based on such test results. In one example, a medical professional determines a low light therapy dosing regimen for the patient based on results of one or more ophthalmic tests. In another example, a machine learning model is used to automatically determine a low light therapy dosing regimen for the patient based on results of one or more ophthalmic tests, such as being trained through a supervised learning process using training data that is based on clinical outcomes of past low light therapy dosing regimens for patients that are associated with particular ophthalmic test results. Dosing information determined based on test results may be transmitted to the headset for use in administering low light therapy to the patient. Furthermore, after low light therapy has been administered, one or more ophthalmic tests may be administered to the patient to determine effects of administering low light therapy, and results of such testing may be transmitted to the remote system for use in determining updated low light therapy dosing information for the patient. The updated low light therapy dosing information may be transmitted to the headset for use in administering low light therapy to the patient (e.g., thus creating an interactive feedback loop by which dosing information is regularly updated based on changes in the patient's condition).

Embodiments of the present disclosure accomplish various technical improvements. For example, configuring a headset to confirm an identity of a patient prior to administering low light therapy to the patient prevents unauthorized or incorrect use of such functionality, which would otherwise be possible with existing techniques. Automatically limiting administration of low light therapy to amounts, types, and/or frequencies indicated in prescribed doses (e.g., received from a medical professional) for a particular patient allows a headset to be used in a safe and customizable manner to administer low light therapy even outside of a medical office, such as in a home environment, and even across multiple patients with different prescribed doses. Generating notifications in an automated manner when low light therapy doses are due, such as providing notifications to a device (e.g., mobile phone) associated with a patient, prevents missed doses and enhances the usability and convenience of a headset configured as described herein.

Configuring a headset as described herein to perform one or more ophthalmic tests on a patient enables intelligent generation of patient-specific low light therapy dosing regimens based on up-to-date information about a patient's condition both before and after administration of low light therapy. Performing such tests on the same device used for low light therapy administration enhances convenience and efficiency of assessing a patient's condition in connection with generation and administration of low light therapy dosing regimens. For example, making use of ophthalmic testing technology to capture and identify information related to the ophthalmic condition of a patient both prior to and after low light therapy administration allows techniques described herein to be dynamically informed regarding the clinical effects of particular dosing regimens in particular cases in a manner that is more accurate and objective than would be possible without the use of such testing technology. Additionally, by determining optimal low light therapy dosing regimens that are targeted for a particular patient's attributes and condition, techniques described herein avoid unnecessarily utilization of device resources in connection with performing low light therapy treatments that are unlikely to be beneficial to a given patient under a particular set of circumstances. Accordingly, the embodiments described herein improve utilization of resources, such as utilization of devices used in providing low light therapy treatments.

Embodiments of the present disclosure also involve interactive feedback loops by which the administration of low light therapy is continuously improved based on updated information about a patient's condition, such as generating updated dosing regimens and/or retraining a machine learning model based on updated ophthalmic testing data captured after administering low light therapy according to a previous dosing regimen, thereby reducing suboptimal dosing over time and improving the low light therapy administration technology.

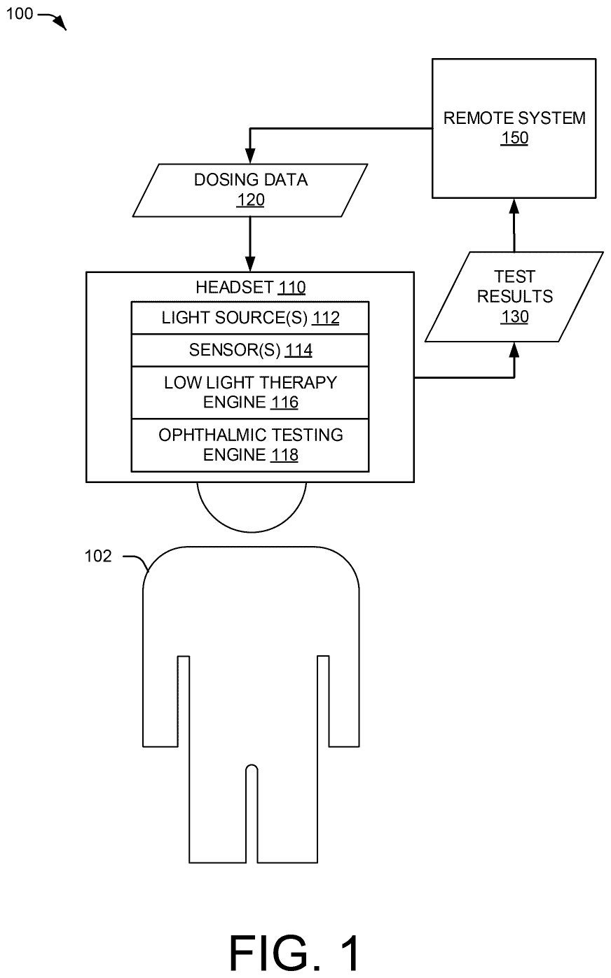

FIG. 1 illustrates an example 100 of components related to administering low light therapy, in accordance with certain embodiments of the present disclosure.

In example 100, a headset 110 is configured to administer low light therapy to a patient 102 via one or more light sources 112. Light source(s) 112 may include one or more lasers, light-emitting diodes (LEDs), and/or broadband light devices, and may be configured to emit light in the visible and/or near infrared spectrum across one or more wavelengths. One or more of light sources 112 may be an LED configured to emit light at one or more wavelengths that stimulate cytochrome —C-Oxidase and/or the Adenosine triphosphate (ATP) energy cycle, such as to improve retina function and/or health. Studies have shown that particular wavelengths, such as 590 nanometers, 660 nanometers, and 850 nanometers, can improve vision by an average of six letters. Light sources 112 may include one or more lasers configured to heat the retinal pigment epithelium (RPE) and create heat shock proteins to improve RPE metabolism and reduce both oxidative stress and inflammation. Such therapy can improve the permeability of Bruchs membrane, reduce thickening, and ultimately redice the size and/or number of drusen deposits (which are a hallmark of retina dysfunction). Such heat shock proteins have been shown to be effective in other diseases, establishing proof of concept for retina use. The laser technology could be Yttrium Aluminum Garnett (YAG) laser technology, frequency double YAG laser technology, femtosecond laser technology, and/or picosecond laser technology. Laser device delivery can be continuous, pulse, or micro-pulse, etc., such as depending on the stage of disease. Further, laser(s), LED(s), and/or superluminescent LED(s) in light source(s) 112 may perform low power therapy for metabolic retina health and/or heat shock protein therapy.

Headset 110 may further comprise one or more sensors 114 configured to capture data related to patent 102. For example, sensor(s) 114 may include one or more cameras, retina and/or iris scanners, fingerprint scanners, autorefractors, keratometers, electrodes, multi-spectral imaging (MSI) devices, and/or the like.

Headset 110 includes a low light therapy engine 116, which may be a software component configured to perform functionality related to administering low light therapy in a safe, controlled, and customizable manner as described herein. For example, low light therapy engine 116 may confirm an identity of patient 102, such as based on data from one or more sensors 114 and/or based on data from one or more other devices (e.g., a mobile phone that includes one or more sensors), and/or based on one or more credentials associated with the patient. In some aspects, headset 110 stores associations between particular patient identifiers and particular biological traits (e.g., relating to a retina, iris, fingerprint, and/or the like) and/or one or more credentials. When low light therapy engine 116 confirms the identity of a patient, low light therapy engine 116 may retrieve low light therapy dosing information (e.g., dosing data 120, received from remote system 150) for that patient, and may administer low light therapy according to such dosing information as appropriate to the patient (e.g., patient 102).

In some aspects, low light therapy engine 116 generates notifications, such as when a dose of low light therapy is due for patient 102 according to dosing data 120, such as providing a notification to a separate device (e.g., mobile phone) associated with the patient and/or displaying such a notification on headset 110. In other embodiments, an application running on a separate device (e.g., mobile phone) that is associated with low light therapy engine 116 may generate such notifications. Thus, patient 102 may be notified when a dose of low light therapy is due soon, due currently, or past due. Furthermore, low light therapy engine 116 may generate notifications for display on headset 110 and/or for transmission to a separate device indicating when new dosing information has been received, when a patient has attempted to log in or be authenticated, when identity of a patient is successfully confirmed or when access is denied (e.g., when an identity of a patient cannot be confirmed), and/or the like.

In certain embodiments, headset 110 is a wearable device, such as being configured to be worn on a patient's head, and includes a display and/or one or more other light sources 112 by which low light therapy is administered to the patient. For example, low light therapy engine 116 may administer low light therapy to patient 102 according to a particular low light therapy dosing regimen for patient 102 (e.g., based on confirming the identity of patient 102), such as only administering low light therapy in amounts, types, and/or frequencies indicated in dosing information for patient 102. For example, headset 110 may prevent administration of low light therapy to a user that is not identified as a patient associated with a low light therapy dosing regimen and/or may prevent administration of low light therapy to a patient that does not have a dose of low light therapy currently due to be administered according to dosing information. In some cases, headset 110 utilizes one or more of Yttrium Aluminum Garnett (YAG) laser technology, femtosecond laser technology, and/or picosecond laser technology to administer low light therapy.

Ophthalmic testing engine 118 may perform one or more ophthalmic tests on patient 102. For example, ophthalmic testing engine 118 may utilize one or more sensors 114 to perform one or more of visual acuity, contrast sensitivity, dark adaptation, optical coherence tomography (OCT), micro-perimetry, scanning laser ophthalmoscopy, multi-spectral imaging (MSI), and/or the like. Results of such ophthalmic testing may be sent as test results 130 to remote system 150 for use in generating an updated dosing regimen. For example, remote system 150 may send updated dosing data 120 indicative of such an updated dosing regimen to headset 110 for use in administering subsequent low light therapy doses.

Remote system 150 may be a computing device associated with a medical professional. Remote system 150 may be connected to headset 110 via a network, such as the Internet or any type of connection over which data may be transmitted. For example, a medical professional may provide input to remote system 500 specifying a low light therapy dosing regimen for patient 102, such as based on test results 130 and/or other information about patient 102. In other embodiments, remote system 150 may utilize a machine learning model to automatically generate a low light therapy dosing regimen for patient 102, such as based on test results 130 and/or other information about patient 102. In one example, such a machine learning model is provided with patient attributes, results of one or more ophthalmic tests, and/or one or more multi-spectral images (MSIs) of the patient as inputs. The machine learning model may have been trained through a supervised learning process to output recommended low light therapy dosing regimens for particular patients based on attributes, test results, and/or MSI(s) related to the patients. The machine learning model may, for example, include a neural network, deep neural network (DNN), convolution neural network (CNN), recurrent neural network (RNN), region-based CNN (R-CNN), long short term memory (LSTM) model, autoencoder (AE) or other type of neural network, a tree-based model (e.g., random forest, gradient boosted tree model, and/or the like), a support vector machine, a generative adversarial network (GAN) model, a logistic regression model, and/or the like. In some embodiments, the machine learning model comprises a single model, while in other embodiments, machine learning model comprises multiple models, such as an ensemble of models. In one particular implementation, the machine learning model comprises a computer vision model or layer such as a CNN for processing images (e.g., MSI(s)), such as outputting patient condition attributes extracted from such images, and an additional model or layer(s) that generates dosing recommendations based on outputs from the computer vision model and additional inputs (e.g., other patient attributes). For example, a machine learning model may accept MSI(s) as inputs, and may extract attributes from MSI(s), such as the quantity, size, and location of particular biomarkers (e.g., drusen, geographic atrophy instances, and/or the like), and may use these extracted attributes along with patient attributes and/or other test results to generate a recommended dosing regimen. Alternatively, the machine learning model may not explicitly extract any features from MSI(s), and may simply use MSI(s) themselves as features when generating a recommended dosing regimen.

In some embodiments, labeled training data such as including sets of input features (e.g., subsets of patient attributes and pre-treatment MSI(s) and/or test results) labeled with dosing regimens that resulted in positive outcomes (e.g., as positive training examples) and/or labeled with dosing regimens that resulted in negative outcomes (e.g., as negative training examples) is used in a supervised learning process to train a machine learning model. In a typical supervised learning process, a set of training inputs is provided to a model, the model generates an output in response to the set of training inputs, the generated output is compared to a label associated with the training inputs, and one or more parameters of the model are adjusted based on the comparing, such as iteratively until one or more conditions are met. For instance, the one or more conditions may relate to an objective function (e.g., a cost function), or may relate to whether the outputs produced by the model based on the training inputs match the labels associated with the training inputs or whether a measure of error between training iterations is not decreasing or not decreasing more than a threshold amount. The conditions may also include whether a training iteration limit has been reached. Parameters adjusted during training may include, for example, hyperparameters, values related to numbers of iterations, weights, functions used by nodes to calculate scores, and the like. In some embodiments, validation and testing are also performed for a machine learning model, such as based on validation data and test data, as is known in the art. While some embodiments involve a machine learning model running on remote system 150, other embodiments may involve a machine learning model running on headset 110 being used to generate recommended dosing regimens as described herein. In some cases, a recommended dosing regimen generated using a machine learning model (e.g., either on remote system, headset 110, or some other device) is confirmed by a user of remote system 150 (e.g., a medical professional), and remote system 150 sends confirmation of such a recommended dosing regimen to headset 110 (e.g., as part of dosing data 120, or dosing data 120 may inherently indicate such confirmation, as remote system 150 may only send dosing data to headset 110 if the dosing data has been confirmed by a medical professional).

In some embodiments, MSI(s) of patient 102 are captured using an MSI device such as a multi-spectral camera, which may be part of headset 110 (e.g., one of sensor(s) 114) or another device. For example, MSI(s) may include one or more multi-spectral images of the eye of patient 102. Multi-spectral imaging is an approach for imaging the eye within multiple wavelength bands. Typical MSI techniques illuminate the eye with narrow-band light at a plurality of different bands (e.g., across a range of wavelengths), and the light is generally detected by a detector after passing through one or more transmission filters matching the narrow-band light, such as to allow only a subset of wavelengths from the range of wavelengths to pass through. A set of MSIs may be generated based on such detection, each of which may correspond to a specific spectral band. MSIs enable the visualization of a wide array of retinal and choroidal pathologies including retinovascular disorders, retinal pigment epithelial changes, and choroidal lesions. For example, an MSI may include visible biomarkers such as drusen and geographic atrophy that are not visible in other types of images. An MSI cannot be captured or processed mentally, as capturing and processing an MSI involves generating light at multiple different wavelengths, passing the light through one or more particular transmission filters, and detecting the light after such filtering, none of which can be performed in the human mind.

Patient attributes generally includes information about a patient, such as personal characteristics (e.g., age, gender, national origin, and/or the like), medical history (e.g., known medical conditions, information about the extent of known medical conditions, procedures that have been performed on the patient, medications taken by the patient, information about medical conditions of family members, and/or the like), and/or other information about the patient and/or the patient's medical condition. In some embodiments, patient attributes includes information about past low light therapy treatments performed on the patient, such as dosing information (e.g., quantity and frequency of treatments) and/or when (e.g., how recently) such treatment(s) were performed.

A machine learning model may process patient attributes, MSI(s), and/or other ophthalmic test results and, in response, output a recommended dosing regimen. The recommended dosing regimen may indicate an amount, type, and/or frequency of low light (e.g., PBM) treatment recommended for the patient to achieve an optimal result (e.g., to achieve a maximum improvement and/or reduction in deterioration in the patient's condition). Alternatively, a dosing regimen may be input to remote system 150 by a user, such as a medical professional, such as based on MSI(s), patient attributes, other ophthalmic test results, and/or other information about the patient.

Dosing data 120 generally represents a dosing regimen for patient 102, such as based on a recommended dosing regimen output by a machine learning model or a dosing regimen input by a user. Dosing data 120 may indicate one or more types, amounts, and/or frequencies of low light therapy to be administered to patient 102 (e.g., indicating how often low light therapy should be administered). One example of dosing data 120 includes dosing of three wavelengths in nine treatments over a period of five weeks, such as including 40 second treatments at 0.4 Joules per square centimeter (J/cm²) each. Dosing data 120 may be transmitted from remote system 150 to headset 110 via a network such as the Internet or any connection over which data may be transmitted.

Low light therapy may be performed on headset 110 based on dosing data 120. After such treatment (e.g., after one or more of the doses recommended in dosing data 120 have been completed), one or more updated MSIs and/or other test results 130 may be captured. Updated MSI(s) and/or other test results 130 may indicate any changes in the patient's condition (or a lack of change in the patient's condition) that resulted from the low light therapy treatment. For example, the quantity, location, and/or size of one or more biomarkers may have changed between initial MSI(s) and/or other test results 130 and updated MSI(s) and/or other test results 130, and such a change may indicate a change in the patient's condition resulting from treatment. If there is no change in such biomarkers, this may indicate that the patient's condition did not change as a result of low light therapy treatment. Test results 130 may be transmitted from headset 110 to remote system 150 via a network such as the Internet or any connection over which data may be transmitted.

In some embodiments, a machine learning model may be re-trained based on updated MSI(s) and/or other updated test results 130 and/or one or more updated patient attributes. For example, updated training data may be generated based on such data (e.g., indicating a change in the patient's condition) such that the machine learning model is able to learn from the actual outcome of performing low light therapy for the patient according to the configured dosing regimen indicated in dosing data 120. Once re-trained, the machine learning model may be used to generate subsequent recommended dosing regimens for the same patient or different patients with a higher level of accuracy.

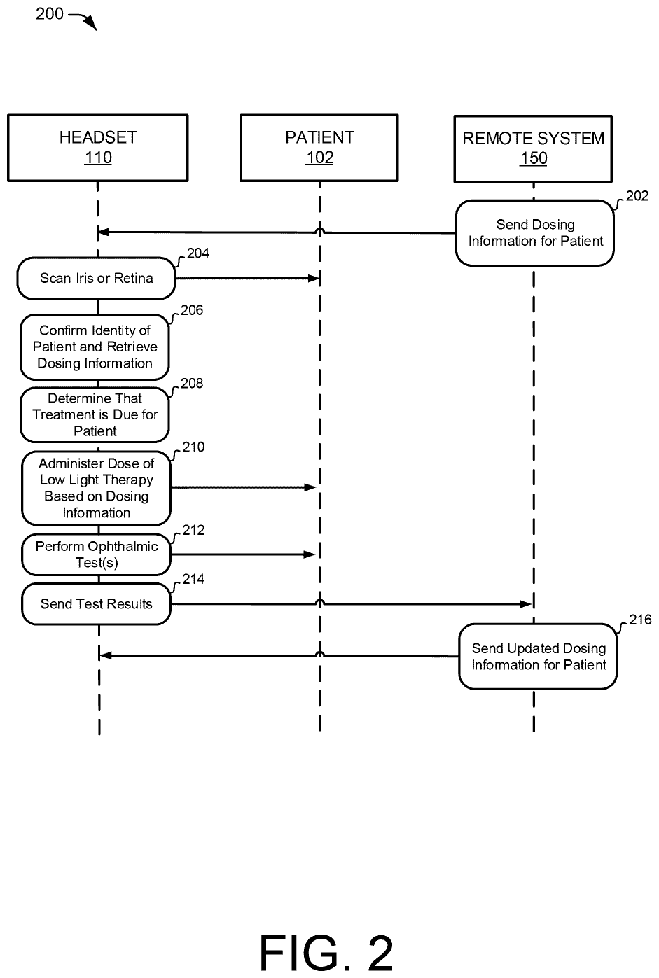

FIG. 2 is a sequence diagram 200 illustrating an example related to administering low light therapy, in accordance with certain embodiments of the present disclosure. Sequence diagram 200 includes headset 110, patient 102, and remote system 150 of FIG. 1.

At 202, remote system 150 sends dosing information for the patient (e.g., patient 102) to headset 110. For example, the dosing information may correspond to dosing data 120 of FIG. 1, described above.

At 204, headset 110 scans the retina or iris of patient 102. In other embodiments, headset 110 scans the fingerprint or other biological characteristic of patient 102. In alternative embodiments, headset 110 receives data from a separate device (e.g., mobile phone) associated with patient 102 that scans the iris, retina, and/or fingerprint of patient 102 and provides results of such scanning to headset 110. In certain embodiments, headset 110 receives one or more credentials of patient 102, such as a username and/or password, which patient 102 may input via headset 110 and/or a separate device.

At 206, headset 110 confirms an identity of patient 102, such as based on the scanning performed at 204 and/or based on other data (e.g., credentials and/or data received from another device) and retrieves dosing information for the patient based upon such confirmation. For example, upon confirming the identity of patient 102, headset 110 may retrieve the dosing information that was sent at 202, such as from one or more memory devices of headset 110.

At 208, headset 110 determines that treatment is due for patient 102, such as based on the dosing information retrieved at 206. For example, headset 110 may determine that a sufficient amount of time has passed since a most recent low light therapy dose was administered to patient 102 (and that another dose is due to be administered) and/or that a first low light therapy dose is due to be administered to patient 102. Alternatively, if headset 110 determines that no dose of low light therapy is due for patient 102, headset 110 may notify patient 102 that no dose is due and may prevent administration of low light therapy until such time as a dose is due for patient 102.

At 210, after determining that treatment is due for patient 102, headset 110 administers a dose of low light therapy to patient 102 based on the dosing information. For example, headset 110 may administer a type and/or quantity of low light therapy indicated in the dosing information for patient 102 via one or more light sources.

At 212, headset 110 performs one or more ophthalmic tests on patient 102. For example, headset 110 may perform one or more of visual acuity, contrast sensitivity, dark adaptation, optical coherence tomography (OCT), micro-perimetry, scanning laser ophthalmoscopy, multi-spectral imaging (MSI), and/or the like on patient 102, such as after administering the dose of low light therapy at 210. In some embodiments, the one or more ophthalmic tests may be performed on the patient before administering the dose of low light therapy (or may even be administered on the patient during a session in which no low light therapy is administered).

In some embodiments, the same test or group of tests may be administered each time the patient uses the headset for low light therapy. In some embodiments, different tests may be performed during different sessions (e.g., to avoid a prolonged test session with all of the doctor ordered tests, the ordered tests may be spread out over several low light therapy sessions). In some embodiments, the tests may be repeated each time low light therapy is administered. In some embodiments, the tests may be repeated at a doctor specified interval (e.g., once a day, once a week, once a month, etc.)

At 214, headset 110 sends results of the performing of the one or more ophthalmic tests (e.g., at 212) to remote system 150. Remote system 150 may generate (e.g., using a machine learning model or other algorithm) and/or receive (e.g., from a medical professional) updated dosing information for patient 102 based on the test results.

In some embodiments, the tests may also be analyzed (remotely or locally on the headset) to determine if there has been a decline in patient vision (e.g., over a predetermined threshold in a predetermined time period). If so, the system may alert a doctor (e.g., that a visual decline greater than, e.g., five percent in one week, has occurred in the patient). Other thresholds and time periods are also contemplated (e.g., greater than five percent in one month, greater than 1 % in one day, etc.) In some embodiments, the analysis may be used to generate reports indicative of the test results that are provided to the doctor and/or patient. In some embodiments, information from the tests may be conveyed to the patient through the headset (or, for example, a mobile device).

At 216, remote system 150 sends updated dosing information for patient 102 to headset 110. The updated dosing information may be used by headset 110 to administer one or more subsequent doses of low light therapy to patient 102, such as upon confirming the identity of patient 102 and determining that such doses are due.

It is noted that headset 110 may be used to administer low light therapy doses to multiple patients that share headset 110. For example, multiple members of a household may have accounts with headset 110, and headset 110 may administer an appropriate dose of low light therapy to a given member of the household based on confirming the identity of that member of the household as described herein (e.g., based on a retina, iris, or fingerprint scan and/or based on one or more credentials). Dosing information for each patient may be transmitted to headset 110 (e.g., from remote system 150) and stored at headset 110 in association with an identifier of that patient. Furthermore, headset 110 may perform ophthalmic testing on multiple patients based on confirming identities of individual patients in a similar manner.

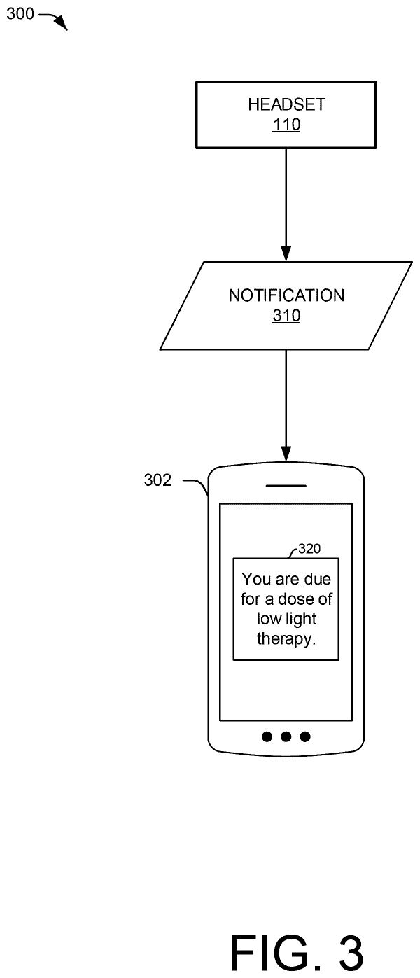

FIG. 3 illustrates an example 300 of an electronic notification related to administering low light therapy, in accordance with certain embodiments of the present disclosure. Example 300 includes headset 110 of FIGS. 1 and 2.

In example 300, headset 110 sends a notification 310 to a device 302. For example, device 302 may be a mobile phone or other type of computing device used by patient 102 of FIGS. 1 and 2. A window 320 may be displayed on device 302, such as within an application associated with headset 110, within a messaging application, on a home screen of device 302, and/or the like, including contents of notification 310. For example, window 320 may include text such as “You are due for a dose of low light therapy.” In one example, notification 310 is sent to device 302 via a text message or email, while in another example notification 310 is sent to device 302 within a particular application. Window 320 may be automatically displayed on device 302 upon receipt of notification 310, and/or may be displayed when a user of device 302 opens notification 310.

Other types of notifications may also be provided by headset 110 to device 302. For example, other such notifications may include indications that a certain patient or patients have attempted to log in to headset 110, that a particular patient or patients were successfully or unsuccessfully authenticated (e.g., a patient may be successfully authenticated if the identity of the patient is confirmed), that a low light therapy dose is due soon or past due, that a low light therapy dose was administered, that updated dosing information has been received by headset 110 for the patient, that updated ophthalmic test results for the patient are available, and/or the like.

In some embodiments, notifications such as notification 310 are sent to device 302 from a separate component, such as remote system 150 of FIGS. 1 and 2, an application server associated with headset 110, and/or the like.

Device 302 may further include one or more sensors, such as a camera, retina scanner, iris scanner, fingerprint scanner, and/or the like, and may be used to scan one or more biological characteristics of a patient for use in confirming the identity of the patient. For example, results of such a scan may be sent from device 302 to headset 110, and headset 110 may confirm an identity of a patient based on such results.

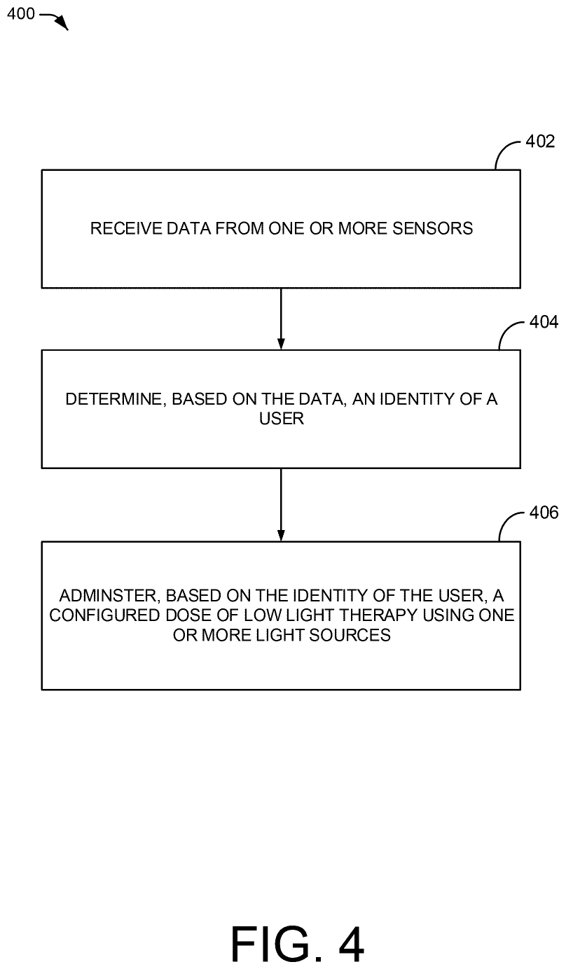

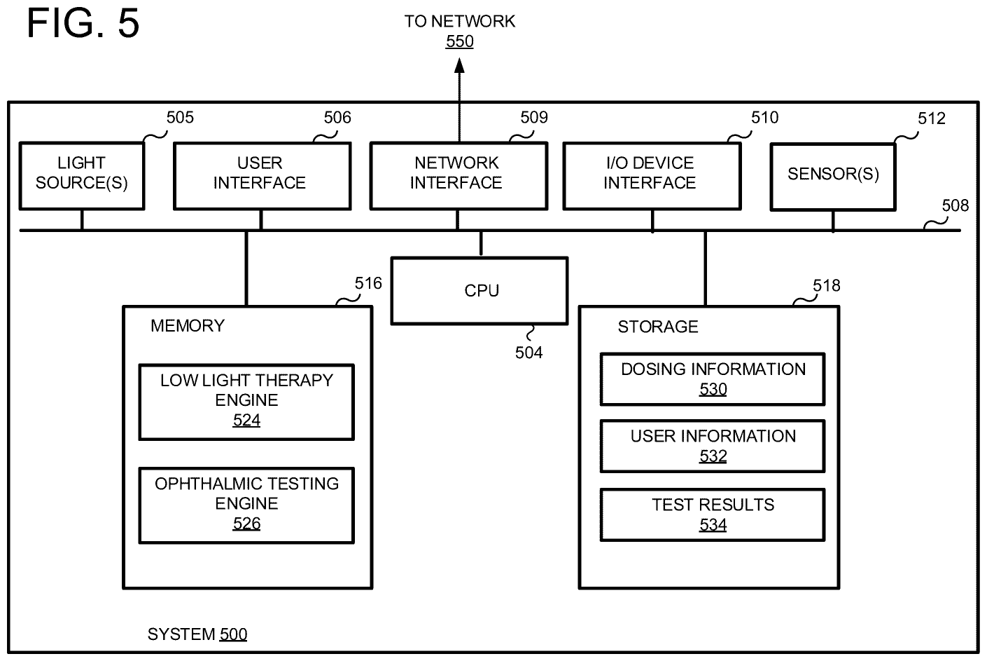

FIG. 4 illustrates an example of a process 400 related to administering low light therapy, in accordance with certain embodiments of the present disclosure. In certain embodiments, the process 400 can be implemented by one or more components described above with respect to FIGS. 1-3 and/or below with respect to FIG. 5. It is noted that any number of systems, in whole or in part, can implement the process 400. In one embodiment, process 400 is performed by headset 110 of FIGS. 1-3.

Process 400 begins at block 402, with receiving data from one or more sensors. For example, the data may be received by headset 110 of FIGS. 1-3 from one or more sensors integrated with the headset or one or more sensors integrated with a separate device.

In certain embodiments, the data from the one or more sensors comprises retina scan data or iris scan data.

Process 400 continues at block 404, with determining, based on the data from the one or more sensors, an identity of the user.

Process 400 continues at block 406, with administering, based on the identity of the user, a configured dose of low light therapy using one or more light sources. For example, the one or more light sources may be integrated with the headset. In some embodiments, the one or more light sources comprise one or more of: a light emitting diode (LED); or a laser.

In some embodiments, the configured dose comprises a type and duration of light therapy associated with the identity of the user.

In certain embodiments, the administering of the configured dose of low light therapy using the one or more light sources is based on determining that a given amount of time has passed since a previous dose of low light therapy was administered to the user.

Some embodiments further comprise transmitting, to one or more endpoints associated with the user, a notification that the user is due for the configured dose of low light therapy.

In some embodiments, the configured dose of low light therapy comprises a photo-bio-modulation (PBM) treatment. In some embodiments, the one or more wavelengths comprise one or more of: 590 nanometers; 660 nanometers; or 850 nanometers.

In certain embodiments, the configured dose is administered to one or both eyes of the user via a headset.

In some embodiments, the administering of the configured dose of low light therapy using the one or more light sources comprises using one or more of: Yttrium Aluminum Garnett (YAG) laser technology; femtosecond laser technology; or picosecond laser technology.

Some embodiments further comprise receiving the configured dose, in association with the identity of the user, from a device associated with a healthcare provider.

Certain embodiments further comprise performing, by the system, an ophthalmic test on the user after the administering of the configured dose. In some embodiments, the performing of the ophthalmic test comprises performing one or more of: a visual acuity test; a dark adaptation test; an optical coherence tomography (OCT) test; a micro-perimetry test; a multi-spectral imaging (MSI) test; or a scanning laser ophthalmoscopy test.

Certain embodiments further comprise transmitting results of the performing of the ophthalmic test to a device associated with a healthcare provider.

Some embodiments further comprise determining an updated dose of low light therapy for the user based on results of the performing of the ophthalmic test. In certain embodiments, the determining of the updated dose of low light therapy for the user based on the results of the performing of the ophthalmic test is based on using a machine learning model. Some embodiments further comprise receiving confirmation of the updated dose of low light therapy from a device associated with a healthcare provider.

Certain embodiments further comprise determining, based on additional data from the one or more sensors, an identity of a different user and administering, based on the identity of the different user, a different configured dose of low light therapy using the one or more light sources.

FIG. 5 illustrates an example of a system 500 for administering low light therapy, in accordance with certain embodiments of the present disclosure. For example, system 500 may be configured to perform process 400 of FIG. 4 and/or other aspects of the present disclosure, such as discussed above with respect to FIGS. 1-3.

As shown, system 500 includes, without limitation, central processing unit (CPU) 504, user interface 506, network interface 509, memory 516, storage 518, interconnect 508, light source(s) 505, sensor(s) 512, and at least one I/O-(Input/Output) device interface 510 which may allow for the connection of various I/O devices (e.g., keyboards, displays, mouse devices, pen input, etc.) to system 500. While one or more operations are described herein as being performed by particular components of system 500, those operations may, in some embodiments, be performed by other components of system 500 and/or component(s) of other system(s). As an example, while one or more operations are described herein as being performed by CPU 504, memory 516, and/or storage 518 those operations may, in other embodiments, be performed by other components of system 500 or of a different system.

CPU 504 may be representative of one or more processing devices and/or cores. In some embodiments, CPU 504 may retrieve and execute programming instructions stored in memory 516. Similarly, CPU 504 may retrieve and store application data residing in memory 516. Interconnect 508 transmits programming instructions and application data, among CPU 504, I/O device interface 510, user interface 506, memory 516, storage 518, network interface 509, etc. In some embodiments, CPU 504 may correspond to a single CPU, multiple CPUs, or a single CPU having multiple processing cores. Additionally, in some embodiments, memory 516 represents volatile memory, such as random-access memory. In some embodiments, storage 518 may be non-volatile memory, such as a disk drive, solid state drive, or a collection of storage devices distributed across multiple storage systems.

System 500 can include a network interface 509 for connection with a data communications network (e.g., network 550), such as to communicate with other devices. The data communications network can be, or can include, one or more of a private network, a public network, a local or wide area network, the Internet, combinations of the same, and/or the like. The data communications network can include, for example, interfaces (e.g., application programming interfaces) for enabling interaction and communication between and among the components and systems of the computing environment (e.g., of FIG. 3) and/or other components and systems.

The memory 516 can include a low light therapy engine 524 and an ophthalmic testing engine 526, which may generally represent low light therapy engine 116 and ophthalmic testing engine 118 of FIG. 1.

The storage 518 can include dosing information 520, which may include dosing data 120 of FIG. 1, the dosing information sent at 202 of FIG. 2, and/or the like, and may include dosing information for one or more patients associated with identifiers of the one or more patients. The storage 518 can further include user information 532, which may include information about users such as patients, and may include patient identifiers, information for use in confirming identities of patients (e.g., biological characteristics and/or credentials), and/or associations between users (e.g., patients) and particular dosing data, such as including records of past low light therapy doses administered to the patients (e.g., including types, amounts, and times of such administered doses in association with patient identifiers). The storage 518 can further include test results 534, which may include test results 130 of FIG. 1 and/or test results determined at 212 and sent at 214 of FIG. 2.

Light source(s) 505 may be representative of light source(s) 112 of FIG. 1. Sensor(s) 512 may be representative of sensor(s) 114 of FIG. 1.

It is noted that system 500 is included as an example, and techniques described herein may be implemented via fewer or more components, either on the same or different devices, and devices may include physical and/or virtual devices.

As used herein, a phrase referring to “at least one of” a list of items refers to any combination of those items, including single members. As an example, “at least one of: a, b, or c” or “at least one of: a, b, and c” is intended to cover a, b, c, a-b, a-c, b-c, and a-b-c, as well as any combination with multiples of the same element (e.g., a-a, a-a-a, a-a-b, a-a-c, a-b-b, a-c-c, b-b, b-b-b, b-b-c, c-c, and c-c-c or any other ordering of a, b, and c).

The foregoing description is provided to enable any person skilled in the art to practice the various embodiments described herein. Various modifications to these embodiments will be readily apparent to those skilled in the art, and the generic principles defined herein may be applied to other embodiments. Thus, the claims are not intended to be limited to the embodiments shown herein, but are to be accorded the full scope consistent with the language of the claims.

Within a claim, reference to an element in the singular is not intended to mean “one and only one” unless specifically so stated, but rather “one or more. ” Unless specifically stated otherwise, the term “some” refers to one or more. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112(f) unless the element is expressly recited using the phrase “means for” or, in the case of a method claim, the element is recited using the phrase “step for.” The word “exemplary” is used herein to mean “serving as an example, instance, or illustration.” Any aspect described herein as “exemplary” is not necessarily to be construed as preferred or advantageous over other aspects.

What is claimed is:

1. A system for administering low light therapy, the system comprising:
one or more light sources configured to generate outgoing light across a range of wavelengths;
one or more sensors configured to scan a biological attribute of a user; and
one or more processors configured to execute instructions that cause the system to:
determine, based on data from the one or more sensors, an identity of the user; and
administer, based on the identity of the user, a configured dose of low light therapy using the one or more light sources.

2. The system of claim 1, wherein the one or more light sources comprise one or more of:
a light emitting diode (LED); or
a laser.

3. The system of claim 1, wherein the data from the one or more sensors comprises retina scan data or iris scan data.

4. The system of claim 1, wherein the configured dose comprises a type and duration of light therapy associated with the identity of the user.

5. The system of claim 1, wherein the administering of the configured dose of low light therapy using the one or more light sources is based on determining that a given amount of time has passed since a previous dose of low light therapy was administered to the user.

6. The system of claim 1, wherein the one or more processors are further configured to execute additional instructions that cause the system to transmit, to one or more endpoints associated with the user, a notification that the user is due for the configured dose of low light therapy.

7. The system of claim 1, wherein the configured dose of low light therapy comprises a photo-bio-modulation (PBM) treatment.

8. The system of claim 1, wherein the one or more wavelengths comprise one or more of:
590 nanometers;
660 nanometers; or
850 nanometers.

9. The system of claim 1, wherein the system comprises a headset, and wherein the configured dose is administered to one or both eyes of the user via the headset.

10. The system of claim 1, wherein the administering of the configured dose of low light therapy using the one or more light sources comprises using one or more of:
Yttrium Aluminum Garnett (YAG) laser;
femtosecond laser; or
picosecond laser.

11. The system of claim 1, wherein the one or more processors are further configured to execute additional instructions that cause the system to receive the configured dose, in association with the identity of the user, from a device associated with a healthcare provider.

12. The system of claim 1, wherein the one or more processors are further configured to execute additional instructions that cause the system to perform, by the system, an ophthalmic test on the user after the administering of the configured dose.

13. The system of claim 12, wherein the performing of the ophthalmic test comprises performing one or more of:
a visual acuity test;
a dark adaptation test;
an optical coherence tomography (OCT) test;
a micro-perimetry test;
a multi-spectral imaging (MSI) test; or
a scanning laser ophthalmoscopy test.

14. The system of claim 12, wherein the one or more processors are further configured to execute additional instructions that cause the system to transmit results of the performing of the ophthalmic test to a device associated with a healthcare provider.

15. The system of claim 12, wherein the one or more processors are further configured to execute additional instructions that cause the system to determine an updated dose of low light therapy for the user based on results of the performing of the ophthalmic test.
